(19) United States
(12) Patent Application Publication
(10) Pub. No.: US 2022/0323143 A1
(43) Pub. Date: Oct. 13, 2022

(54) DEPLOYABLE STRUCTURES TO PROVIDE ELECTRODES ON THE SURFACE OF AN ENDOSCOPICALLY GUIDED LASER ABLATION CATHETER FOR USE IN ABLATION AND ELECTROPHYSIOLOGICAL MAPPING

(71) Applicant: CardioFocus, Inc., Marlborough, MA (US)

(72) Inventors: Gerald Melsky, Lexington, MA (US); Brian Estabrook, Lenox, MA (US); Burke Barrett, Franklin, MA (US); Jeff Brown, North Andover, MA (US); Lincoln Baxter, Centerville, MA (US); Kevin Zephir, Westminster, MA (US); Curtis Keohane, Marlborough, MA (US); Omar Colon, Nashua, NH (US)

(21) Appl. No.: 17/711,574

(22) Filed: Apr. 1, 2022

Related U.S. Application Data
(60) Provisional application No. 63/169,437, filed on Apr. 1, 2021.
(60) Provisional application No. 63/238,821, filed on Aug. 31, 2021.
(60) Provisional application No. 63/312,684, filed on Feb. 22, 2022.
(60) Provisional application No. 63/314,010, filed on Feb. 25, 2022.

(51) Int. Cl. A61B 18/14; A61B 18/12

(52) U.S. Cl. CPC A61B 18/1492; A61B 18/1206; A61B 2018/0022

(57) ABSTRACT

A method for ablating target tissue includes the steps of: (a) delivering an ablation balloon catheter to the target tissue, wherein the ablation balloon catheter includes a compliant balloon, a visualization device; and an electrode array that is visible to the visualization device, each electrode being configured to deliver ablation energy, wherein the electrode array is independently movable relative to the compliant balloon; (b) isolating the target tissue such that at least one electrode of the electrode array is in contact with the target tissue; and (c) delivering the ablation energy to those electrodes of the electrode array that are confirmed, using the visualization device, to be in contact with target tissue.

### CROSS REFERENCE TO RELATED APPLICATIONS

The present application claims priority to and the benefit of U.S. patent application Ser. No. 63/169,437, filed Apr. 1, 2021; U.S. patent application Ser. No. 63/238,821, filed Aug. 31, 2021; U.S. patent application Ser. No. 63/312,684, filed Feb. 22, 2022; and U.S. patent application Ser. No. 63/314,010, filed Feb. 25, 2022, each of which is hereby expressly incorporated by reference in its entirety.

### TECHNICAL FIELD

The present disclosure is directed to ablation of atrial fibrillation and specifically to ablation of atrial fibrillation with a device that includes a deployable structure to provide electrodes on the surface of an endoscopically guided laser ablation catheter for use in ablation and electrophysiological mapping.

### BACKGROUND

Balloon catheters that are configured to perform ablation of atrial fibrillation are well known and are described in Melsky et al U.S. Pat. No. 9,421,066B2 and Melsky et al U.S. Pat. No. 9,033,961B2, each of which is hereby expressly incorporated by reference in its entirety. The aforementioned patents treat atrial fibrillation by using an energy source to create non-electrically conducting lesions in the atrial tissue in such a fashion that a circumferential ring of lesion is created in the region of the left atrium where the pulmonary veins join the atrium. Such circumferential lesions prevent electrical signals originating in the veins from entering the atrium and vice versa. Blocking the passage of such electrical signals can, in most cases, restore sinus rhythm to a previously fibrillating left atrium.

Typically, ablation for atrial fibrillation consists of the steps of introducing an ablation catheter into the left atrium, creating the circumferential lesions around the pulmonary veins and then confirming that the circumferential lesions have been adequately produced so as to actually block electrical signals. This confirmation process generally consists of removing the ablation catheter then introducing a catheter with multiple electrodes which can be placed in a pulmonary vein distal to the circumferential lesion and then using the electrodes to monitor the electrograms originating in the pulmonary veins. When the vein has been electrically isolated from the atrium, the vein is silent with only far-field electrical activity seen in the vein. Occasional spikes within the vein may occur but with no conduction to the rest of the atrium. Pacing the atrium via a catheter with electrodes placed in the coronary sinus can help confirm that only far-field activity and random spikes are seen in the vein.

Now, the aforementioned devices in Melsky et al U.S. Pat. No. 9,421,066B2 and Melsky et al U.S. Pat. No. 9,033,961B2 are effective ablation devices but, as with many other ablation devices, they contain no means for quickly and easily confirming electrical isolation once the ablation of a vein has been completed. It is very desirable to be able to ablate veins and then, without having to exchange catheters, be able to confirm that the ablation has resulted in the desired electrical isolation of the veins. Therefore, one object of the present invention is to provide an ablation device that provides endoscopically guided laser ablation and provides a means to confirm that electrical isolation of the pulmonary veins has been achieved and to perform such confirmation without the need to remove or exchange catheters. Exchanging catheters carries the risk of introducing air into left atrium if performed incorrectly. Air introduction into the left atrium could lead to damage to the brain or heart or of other organs should the air travel into the organs capillary beds and impede blood flow there. For this reason, catheter exchanges are always done slowly and methodically to minimize the risk of air introduction. However, slow and methodical catheter exchanges increase the time to complete an ablation procedure. Prolonged procedures carry other risks to the patient as well as increasing the cost of the procedure so reducing the number of catheter exchanges during a procedure is desirable.

In addition to confirming electrical isolation of the veins has been achieved, the addition of electrodes to the ablation catheters described in Melsky et al U.S. Pat. No. 9,421,066B2 and Melsky et al U.S. Pat. No. 9,033,961B2 would also enable the delivery of ablative energy that requires an electrically conductive pathway from the energy source to the region of ablation. The ablative energy delivered may be either radiofrequency energy or electroporative energy (also called pulsed field ablation energy) or other energy, such as laser or microwave. The ability to deliver these other ablative energy types may be desirable in instances where anatomical considerations favor one type of energy over the other. For example, laser energy is desirable because it creates lesions that penetrate through the full thickness of the atrial wall thus ensuring that the electrical disassociation caused by lesions created using laser energy will be robust and durable. However in circumstances where the esophagus lies against the left atrium in an area that must be ablated, use of electroporative energy in that particular region may be desirable since it has been proposed that electroporative energy creates lesions differentially in cardiac tissue and esophageal tissue thereby opening the possibility that cardiac tissue adjacent to the esophagus can be safely ablated via electroporation without the need to closely monitor the temperature of the esophagus and halt ablation if the esophagus temperature rises too high.

### SUMMARY

In summary, one object of the present disclosure is to provide a means to quickly and easily confirm electrical isolation of pulmonary veins that have been isolated by endoscopically guided laser ablation using devices similar to those described in Melsky et al U.S. Pat. No. 9,421,066B2 and Melsky et al U.S. Pat. No. 9,033,961B2. A further object of the invention is to provide such means in such a manner that no catheter exchanges are required. A further object of the invention is to provide a means to both confirm isolation and to deliver other forms of ablative energy that can be delivered via electrodes which either contact the tissue or are in close proximity to tissue. A further object of the invention is to provide electrodes for either isolation confirmation or ablation that can be visualized endoscopically using the endoscopic apparatus already present in Melsky et al U.S. Pat. No. 9,421,066B2 and Melsky et al U.S. Pat. No. 9,033,961B2.

In one exemplary embodiment, a method for ablating target tissue includes the steps of:

delivering an ablation balloon catheter to the target tissue, wherein the ablation balloon catheter includes a compliant balloon, a visualization device; and an electrode array that is visible to the visualization device, each electrode being configured to deliver ablation energy, wherein the electrode array is independently movable relative to the compliant balloon;

isolating the target tissue such that at least one electrode of the electrode array is in contact with the target tissue; and

delivering the ablation energy to those electrodes of the electrode array that are confirmed, using the visualization device, to be in contact with target tissue.

### BRIEF DESCRIPTION OF DRAWING FIGURES

FIG. 1 shows one exemplary device of the present disclosure in a deployed state, deployed over a surface of an inflated balloon of an exemplary balloon catheter;

FIG. 2 shows the device of FIG. 1 in a retracted state ready to be advanced over a deflated balloon of the balloon catheter;

FIG. 3 shows the device of FIG. 1 in a state in which it has been advanced over an inflated balloon of a balloon catheter and it is in a partial state of deployment, such deployment being accomplished by inflation of the balloon;

FIG. 4 shows a PFA catheter mounted basket;

FIG. 5 shows an electrode catheter for use with a balloon catheter;

FIG. 6 shows a dual transeptal/second catheter device that includes the electrode catheter of FIG. 5 disposed over the balloon catheter;

FIG. 7 shows a retractable tine electrode array embodiment;

FIGS. 8A-8C show the various states of the retractable tine electrode array;

FIG. 9 shows a balloon catheter with PFA braided wire mesh electrode array;

FIG. 10 shows a balloon with embedded electrode array;

FIG. 11 shows balloon catheter with micropores with an inner electrode array;

FIG. 12 shows the balloon catheter with micropores with the inner electrode array;

FIG. 13 shows another balloon catheter with micropores and an inner electrode array; and

FIG. 14 is a block diagram depicting exemplary components of an endoscope-guided cardiac ablation system according to the invention.

### DETAILED DESCRIPTION OF CERTAIN EMBODIMENTS

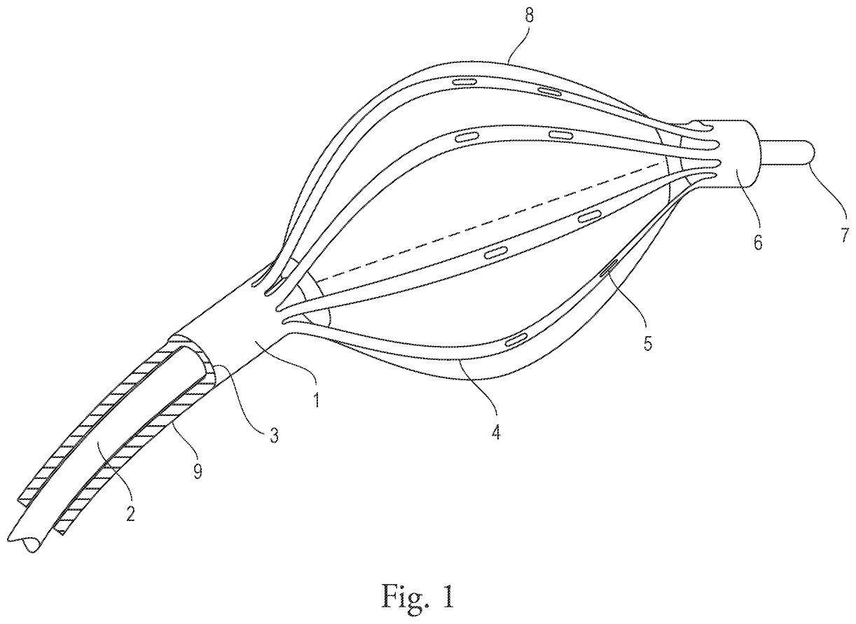

FIG. 1 shows an exemplary balloon catheter, such as the one described in Melsky et al U.S. Pat. No. 9,421,066B2 and Melsky et al U.S. Pat. No. 9,033,961B2, each of which has been incorporated by reference.

Exemplary Ablation System

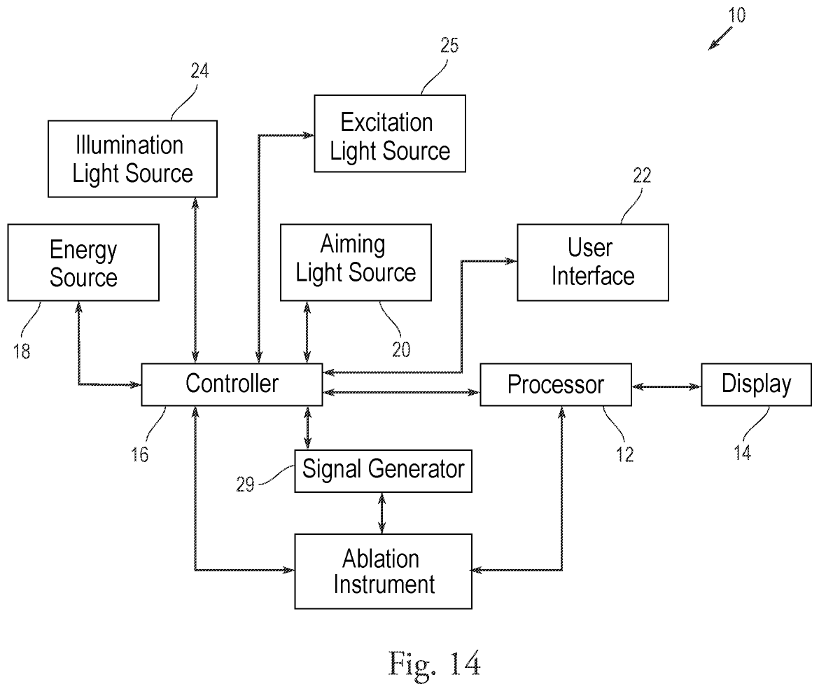

FIG. 14 is an exemplary schematic block diagram illustrating one ablation/endoscopic system in accordance with the invention, designated generally by reference numeral 10. Ablation system 10 preferably includes a treatment ablation instrument, such as one of the ones described herein, preferably including an endoscope and ablation device as discussed below.

The ablator system 10 further preferably includes an aiming light source 20 and an illumination light source 24. A processor 12 designed to accept input and output data from the connected instruments, a display 14, and a controller 16 and process that data into visual information.

As will also be appreciated from the below discussion, an endoscope is preferably provided in ablation instrument 100 and has the capability of capturing both live images and recording still images. An illumination light 24 is used to provide operating light to the treatment site. The illumination light is of a frequency that allows the user to differentiate between different tissues present at the operating site. An aiming light source 20 is used to visualize the location where energy will be delivered by the ablation instrument 100 to tissue. It is envisioned that the aiming light 20 will be of a wavelength that can be recorded by an image capture device and visible on a display.

The processor 12 can be designed to process live visual data as well as data from the ablation instrument controllers and display. The processor 12 is configured execute a series of software and/or hardware modules configured to interpret, manipulate and record visual information received from the treatment site. The processor 12 can further be configured to manipulate and provide illustrative and graphical overlays and composite or hybrid visual data to the display device.

As seen in FIG. 14, the system 10 further includes the controller 16, an energy source 18, the aiming light source 20 and a user interface 22. Controller 16 is preferably configured to control the output of the energy source 18 and the illumination and excitation sources 24 and 25 of an energy transmitter, as well as being configured to determine the distance and movement of an energy transmitter relative to tissue at an ablation treatment site (as discussed further below). As will also be appreciated from the below discussion, an endoscope is preferably supported by the ablation instrument and captures images that can be processed by the processor 12 to determine whether sufficient ablative energy deliveries have been directed to a specific area of a treatment site. Data obtained from the endoscope includes real-time video or still images of the treatment site as seen from the ablation instrument. As discussed herein, these images/videos can be stored in memory for later use.

The aiming light source 20 is used to visualize the treatment site location where energy will be delivered by the ablation instrument to the target tissue. Preferably, the aiming light source 20 outputs light in a visible region of the electromagnetic spectrum. If a suitable ablation path is seen by the user, the controller 16 can transmit radiant energy, via energy source 18, from the ablation instrument to a target tissue site to effect ablation by lesions. It is to be appreciated that the term “radiant energy” as used herein is intended to encompass energy sources that do not rely primarily on conductive or convective heat transfer. Such sources include, but are not limited to, acoustic, laser, electroporative energy, and electromagnetic radiation sources and, more specifically, include microwave, x-ray, gamma-ray, ultrasonic and radiant light sources. Additionally, the term “light” as used herein is intended to encompass electromagnetic radiation including, but not limited to, visible light, infrared and ultraviolet radiation.

The illumination light source 24 is a light source used to provide proper illumination to the treatment site. The illuminate is configured so that natural biological tones and hues can be easily identifiable by an operator.

The controller 16 can provide the user with the ability to control the function of the aiming light source, the user input devices, and the ablation instrument. The controller 16 serves as the primary control interface for the ablation system. Through the controller 16, the user can turn on and off both the aiming and illumination lights 20, 24. Furthermore the controller 16 possesses the ability to change the illumination and aiming light intensity. The ability to switch user interfaces or display devices is also envisioned. Additionally, the controller 16 gives access to the ablation instrument, including control over the intensity of the discharge, duration and location of ablative energy discharges. The controller 16 can further provide a safety shutoff to the system in the event that a clear transmission pathway between the radiant energy source and the target tissue is lost during energy delivery (e.g., see commonly owned U.S. patent application Ser. No. 12/896,010, filed Oct. 1, 2010, which is hereby incorporated by reference in its entirety).

The controller can be a separate microprocessor based control interface hardware or it can be a portion of a configured as a module operating through a processor based computer system configured to accept and control inputs from various physical devices.

Pulsed Electric Field Ablative Energy

While the technical field of pulsed electric fields for tissue therapeutics continues to evolve, it is generally understood that application of brief high DC voltages to tissue may generate locally high electric fields typically in the range of hundreds of volts per centimeter that disrupt cell membranes by generating pores in the cell membrane. While the precise mechanism of this electrically-driven pore generation or electroporation continues to be studied, it is thought that the application of relatively brief and large electric fields generates instabilities in the lipid bilayers in cell membranes, causing the occurrence of a distribution of local gaps or pores in the cell membrane. This electroporation may be irreversible if the applied electric field at the membrane is larger than a threshold value such that the pores do not close and remain open, thereby permitting exchange of biomolecular material across the membrane leading to necrosis and/or apoptosis (cell death). Subsequently, the surrounding tissue may heal naturally.

Generally, a system, such as the ones described herein, for delivering a pulse waveform to tissue includes a signal generator configured for generating a pulse waveform and an ablation device coupled to the signal generator and configured to receive the pulse waveform. In some embodiments, the ablation device is configured to generate an electric field intensity of between about 200 V/cm and about 1500 V/cm. Accordingly, a system for ablating tissue described herein can include a signal generator and an ablation device having one or more electrodes and an expandable/inflatable member (e.g., balloon) for the selective and rapid application of DC voltage to drive electroporation.

In some embodiments, the voltage pulse waveforms disclosed herein may be hierarchical and have a nested structure.

An irreversible electroporation system as described herein may include a signal generator and a processor configured to apply one or more voltage pulse waveforms to a set of electrodes to deliver energy to a region of interest. In order to deliver the pulse waveforms generated by the signal generator, one or more electrodes of the ablation device may have an insulated electrical lead configured for sustaining a voltage potential of at least about 2500 V without dielectric breakdown of its corresponding insulation at least in one embodiment. In some embodiments, at least some of the electrodes may be independently addressable such that each electrode may be controlled (e.g., deliver energy) independently of any other electrode of the device.

As shown in FIG. 14, the system can include a signal generator 29 that is configured to generate pulse waveforms for irreversible electroporation of tissue, such as, for example, a pulmonary vein. For example, the signal generator 29 can be a voltage pulse waveform generator and be configured to deliver a pulse waveform to one of the ablation devices (ablation instruments) described herein. The processor 12 can incorporate data received from memory to determine the parameters of the pulse waveform to be generated by the signal generator 29, while some parameters such as voltage can be input by a user. The memory can further store instructions to cause the signal generator 29 to execute modules, processes and/or functions associated with the system, such as pulse waveform. For example, the memory can be configured to store pulse waveform for pulse waveform generation.

Some embodiments are directed to pulsed high voltage waveforms together with a sequenced delivery scheme for delivering energy to tissue via sets of electrodes. The signal generator and the processor are capable of being configured to apply pulsed voltage waveforms to a selected plurality or a subset of electrodes of an ablation device.

In one application, a pulsed voltage waveform can be in the form of a sequence of double pulses, with each pulse, such as the pulse being associated with a pulse width or duration. The pulse width/duration can be about 0.5 microseconds, about 1 microsecond, about 5 microseconds, about 10 microseconds, about 25 microseconds, about 50 microseconds, about 100 microseconds, about 125 microseconds, about 140 microseconds, about 150 microseconds, including all values and sub-ranges in between. The pulsed waveform can be defined by a set of monophasic pulses where the polarities of all the pulses are the same (e.g., all positive, as measured from a zero baseline). In some embodiments, such as for irreversible electroporation applications, the height of each pulse or the voltage amplitude of the pulse can be in the range from about 400 volts, about 1,000 volts, about 5,000 volts, about 10,000 volts, about 15,000 volts (e.g., in one application a maximum amplitude of 2500 volts is used), including all values and sub ranges in between. The pulse is separated from a neighboring pulse by a time interval, also sometimes referred to as a first time interval. As examples, the first time interval can be about 1 microsecond, about 50 microseconds, about 100 microseconds, about 200 microseconds, about 500 microseconds, about 800 microseconds, about 1 millisecond including all values and sub ranges in between, in order to generate irreversible electroporation. It will be appreciated that the aforementioned values are only exemplary in nature and are not limiting of the scope of the present invention since values outside the aforementioned ranges can exist for other applications.

Exemplary Ablation Catheter

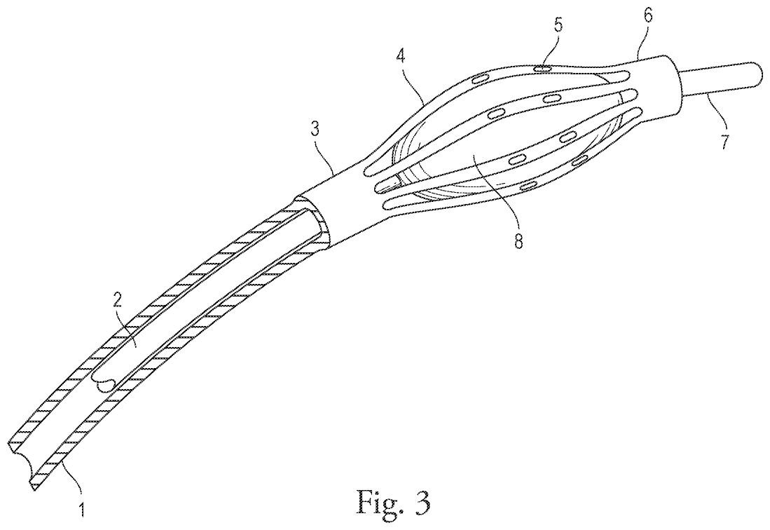

As shown in FIGS. 1-3, one exemplary ablation device is directed to a generally flexible and elongate structure 1, that is slidably disposed over an elongate shaft 2 of a balloon ablation catheter. The elongate structure 1 can be considered to be a sleeve that is longitudinally displaceable over the balloon catheter. While the term “elongate structure” is used herein, it will be understood that the term “sleeve” can be interchangeably used therewith. As described herein, the elongate structure 1 can be moved along the balloon catheter so as to cover different regions of the balloon catheter. As described herein, the elongate structure 1 is configured to respond to the movements of the balloon ablation catheter and more particularly, to the expansion and contraction of the balloon when the elongated structure at least partially covers the balloon.

The elongate structure 1 generally has several different portions including a proximal portion and a distal portion. The proximal portion of the elongate structure 1 comprises a first tubular shaped portion 3 as shown in FIG. 1. This proximal region is spaced back from the distal end at a distance of 2 cm to 4 cm; however, this is merely one exemplary value and not limiting of the scope of the present invention. The first tubular shaped portion 3 is configured such that the shaft 2 of the balloon ablation catheter passes through a lumen of the first tubular shaped portion 3. In other words, the first tubular shaped portion 3 completely surrounds the catheter shaft 2 in at least one region of the first tubular shaped portion 3.

The first tubular shaped portion 3 can be formed of a flexible material.

The distal portion of the elongate structure 1 multifurcates into two or more but preferably six or more branches 4, which are also flexible. Each branch 4 contains one or more electrodes 5 on their outward facing surface. Each electrode 5 is connected to an insulated conductor wire imbedded in the body of the elongate flexible structure 1 but such conductor wires or the like are not shown in FIG. 1. For example, the structure 1 can be overmolded over the conductor wires. As shown, when multiple electrodes 5 are used for each branch 4, the electrodes 5 are spaced longitudinally apart along the respective branch 4. It will also be appreciated that the electrodes 5 can be of the same type or can be different types. In other words, the electrodes 5 can be of different sizes and/or different shapes. The arrangement of the electrodes 5 can be of an asymmetric nature in that the electrodes 5 can be focused on one or more regions of the branches 4. For example, the electrodes 5 can be more centrally located and distally located along the branches 4 as opposed to be located proximally.

The branches 4 can thus be circumferentially spaced apart from one another and extend circumferentially about the balloon. It is also possible for the branches 4 to be designed to have an asymmetric appearance in that instead of having a symmetric angular displacement between the branches 4, an asymmetric arrangement can be provided. In other words, within one half of the elongate structure 1, the branches 4 can have one type of angular displacement and within the other half, a different angular displacement can be provided. In other words, there can be more branches 4 in one half of the structure 1 compared to the other half of the structure 1. For example, the first circumferential half can have a first number of electrodes, while the second circumferential half can have a second number of electrodes that can be different than the first number.

As shown, each branch 4 has a first end (proximal end) and an opposing second end (distal end). The first ends of the branches 4 are attached to the first tubular shaped portion 3 and in one embodiment, the branches 4 are formed integral with the first tubular shaped portion 3.

The multiple flexible branches 4 rejoin at their second ends to again form a second tubular structure 6 at the distal end of the elongate structure 1. The second tubular structure 6 encircles, in a slidable manner (both axially and rotationally), a distal tip 7 of the balloon ablation catheter.

In general, the multifurcations (branches 4) form an expandable cage like structure which circumferentially surrounds the inflated balloon 8 when the elongated structure is positioned over at least a portion of the balloon. The proximal portion of the elongate structure 1 can maintain a tubular shape proximally from the multifurcations (branches 4) on back or, alternatively, the proximal portion of the elongate structure 1 can consist of only a partial circumferential portion of a tube as shown at 9 and thereby be more flexible and occupy less volume than if it were entirely tubular. Shaft 2 can be visible between portions of the elongate structure 1.

It will be appreciated that the present device 1 is preferably formed as a single elongate structure in which the tubular portions 3, 6 and branches 4 located therebetween are formed as a single unitary part (e.g., molded part).

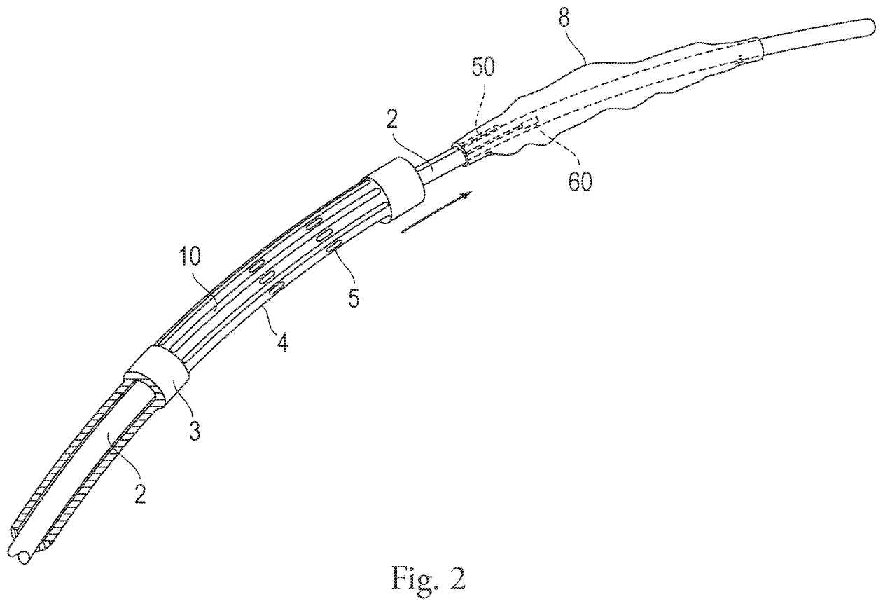

FIG. 2 shows travel of the elongate structure 1 over the balloon catheter. More particularly, the first tubular shaped portion 3 and the branches 4 are shown in their relaxed state. This represents the normal, at rest state of the elongate structure 1. In this state it is clear to see how such a structure can be produced by creating a series of longitudinal slits 10 in a generally thin flat material that has been formed into a tubular shape. In other words, the branches 4 are formed by incorporating longitudinal slits in the structure 1 so as to define one branch between two adjacent slits. A suitable thin flat material would be polyimide film such as is commonly used to produce flexible printed circuits or flex-circuits. It will be appreciated that other materials are equally possible.

FIGS. 1 and 2 together illustrate how the present device accomplishes the objectives of providing a means to allow for pulmonary vein isolation using an endoscopically guided balloon catheter and to additionally provide a means to confirm electrical isolation of the vein without the need to exchange catheters as required in the prior art. As discussed in more detail below, the inner surface of the tubular structure can contain marks on the inside surface that are visible to the endoscope for indicating the location of the electrodes.

There can thus be two defined stages of operation including a first stage which is an ablation stage of the procedure in which the elongate structure 1 is not used. During this ablation stage, the elongate structure 1 resides, as shown in FIG. 2, proximal to the balloon of the balloon catheter and in a collapsed state closely surrounding the shaft 2 of the balloon catheter. As shown in this stage and state, the entire elongate structure 1 is displaced from and located proximal to the balloon of the balloon catheter. The distal second tubular shaft portion 6 is thus located proximal to the balloon.

In this state (first stage), the present elongate structure 1 allows for the balloon of the ablation catheter to be inflated and placed in a pulmonary vein while not being encumbered by the elongate structure 1. The vein may be visualized endoscopically by the ablation catheter and laser energy may be delivered to the vein without regard to the invention. In other words, as in Applicant's previous ablation catheter designs, energy from a movable energy emitter 0 (FIG. 2) that resides within the balloon passes through the balloon to the target site without any impediment from the elongate structure 1 due to the elongate structure 1 being spaced from and not in contact with the inflated operating area of the balloon.

This would not be the case if electrodes (such as electrodes 5) had been placed directly on the surface of the balloon since such electrodes would block both the laser energy and endoscopic visualization over the portion of the balloon on which such electrodes resided.

Once ablation (the first stage) of a vein has been accomplished, the balloon of the ablation catheter is deflated but the elongate structure 1 of the ablation catheter is not repositioned relative to the vein. With the ablation catheter structure stationary relative to the ablated vein, the elongate structure 1 is advanced distally over the deflated balloon. The balloon is then re-inflated and such re-inflation expands the branches (multifurcations) 4 of the elongate structure 1 and forces at least some number of the electrodes 5 into contact with the lumen of the vein. Said electrodes 5 can now be used to confirm electrical isolation by connecting the conductor wires connected to the electrodes 5 and extending proximally along the proximal portion of the elongate structure 1 until they are present outside of the patient's body, to know devices which are capable of amplifying and displaying the electrical activity emanating from the tissue in contact with the electrodes 5.

It should also be noted that the electrodes 5, when in this state of contact with the pulmonary vein tissue (or other target tissue) are also capable of delivering ablative energy such as radiofrequency energy or electroporative energy or microwave energy by connecting a source of such energy to the conductor wire attached to the electrodes. It should be also noted that the positions of the electrodes are visible to the endoscope 50 (FIG. 2) which resides inside the balloon of the ablation catheter. This visibility is accomplished by either making the multifurcations 4 out of a transparent material or by creating marks on the inner surfaces of the multifurcations directly adjacent to the position of the electrodes. Such visualization of the electrode position endoscopically enables a visual assessment of the condition of contact between electrodes and tissue. For example, a given electrode may be in firm contact with the vein tissue throughout the entire cardiac cycle. Alternatively, the electrode 5 can be in contact with tissue during a portion of the cardiac cycle and during the other portion of the cycle, the electrode 5 may not be in contact with tissue but it is in contact with blood instead or the electrode may not be in contact with tissue during any part of the cardiac cycle. Such visual assessments of the nature of the contact between tissue and the electrodes in not currently available in any know devices. Such assessment is valuable in aiding interpretation of the electrograms measured by the electrodes. Further, if the electrodes are to be used for the purposes of applying radiofrequency or electroporative or microwave ablation energy, such visual information about the degree of tissue contact can be used to determine which of the several electrodes are suitable to deliver ablative energy by virtue of the degree of tissue contact they afford. Also, the endoscopic view can be used to guide the repositioning of the balloon in the vein in order to improve the contact between electrodes and the vein tissue if deemed necessary for a better assessment of the electrical activity in the vein or for better electrode contact to enable ablation via radiofrequency or electroporative energy application.

Sliding Action of Elongate Structure 1

As discussed herein, the elongate structure 1 is configured to move longitudinally along the balloon catheter as illustrated in FIGS. 1-3. It is also to have rotationally movement relative to the balloon. The elongate structure 1 can be moved manually as by grasping one end (such as the first tubular portion 3) of the elongate structure 1 and the moving the entire structure 1 longitudinally in a distal or proximal direction. Alternatively, to move the elongate structure 1 in the proximal direction, the first tubular portion 3 can be grasped and pulled in the proximal direction. Preferably, the first tubular portion 3 extend proximally to a point where it exits the body and is available to be grasped directly by the user. To assist the user in moving the structure 1, the most proximal end of the structure 1 can have a grasp feature, such as an enlarged ring section or the like at the proximal end of the first tubular portion 3. Alternatively, surface texture or the like can be provided to one or more regions of the first tubular portion 3.

When the elongate structure 1 is retracted and moved proximally, it can enter into a lumen formed in the catheter structure or into a lumen in a guiding sheath or deflectable sheath commonly employed in atrial ablation procedures, through which the balloon catheter and tubular structure would be passed. This to say that the tubular structure can be slid so that it is retracted into the catheter shaft or into a guiding or deflectable sheath and this retraction will cause the elongate structure 1 to collapse and be removed from surrounding relationship around the balloon. The retraction of the structure 1 within the lumen of the catheter shaft causes the collapsing of branches to a compact state. It is noted that when the tubular structure is retracted into a guiding or deflectable sheath the multifurcations of the tubular structure are supported and prevented from expanding or deflecting outwardly by the inner surface of such sheath and are also prevented from deflecting inwardly by the shaft of the balloon catheter. In such a state the tubular structure is constrained from expanding or contracting and is therefore more easily repositioned relative to the balloon catheter. In the case of a device were the only ablative energy employed is delivered via the electrodes, the elongate structure would not necessarily need to be retracted to a position fully proximal of the balloon. In other words, the elongate structure 1 is movable between a multitude of positions with one position being a position in which at least some electrodes at least partially cover the balloon.

Controllable Electrodes

The overall ablation system described herein that includes the elongate structure 1 and the ablation balloon catheter can communicate over a network to the various machines that are configured to send and receive content, data, as well as instructions that, when executed, enable operation of the various connected components/mechanisms. The content and data can include information in a variety of forms, including, as non-limiting examples, text, audio, images, and video, and can include embedded information such as links to other resources on the network, metadata, and/or machine executable instructions. Each computing device can be of conventional construction, and while discussion is made in regard to servers that provide different content and services to other devices, such as mobile computing devices, one or more of the server computing devices can comprise the same machine or can be spread across several machines in large scale implementations, as understood by persons having ordinary skill in the art. In relevant part, each computer server has one or more processors, a computer-readable memory that stores code that configures the processor to perform at least one function, and a communication port for connecting to the network. The code can comprise one or more programs, libraries, functions or routines which, for purposes of this specification, can be described in terms of a plurality of modules, residing in a representative code/instructions storage, that implement different parts of the process described herein. As described herein, each of the robotic devices (tools) has a controller (processor) and thus, comprises one form of the above-described computing device.

Further, computer programs (also referred to herein, generally, as computer control logic or computer readable program code), such as imaging or measurement software, can be stored in a main and/or secondary memory and implemented by one or more processors (controllers, or the like) to cause the one or more processors to perform the functions of the invention as described herein. In this document, the terms “memory,” “machine readable medium,” “computer program medium” and “computer usable medium” are used to generally refer to media such as a random access memory (RAM); a read only memory (ROM); a removable storage unit (e.g., a magnetic or optical disc, flash memory device, or the like); a hard disk; or the like. It should be understood that, for mobile computing devices (e.g., tablet), computer programs such as imaging software can be in the form of an app executed on the mobile computing device.

The system can include a graphical user interface (GUI) that can be provided to allow for remote control over the system. As is known, the GUI is a system of interactive visual components for computer software. A GUI displays objects that convey information and represent actions that can be taken by the user. The objects change color, size, or visibility when the user interacts with them. GUI objects include icons, cursors, and buttons. These graphical elements are sometimes enhanced with sounds, or visual effects like transparency and drop shadows.

The graphical user interface typically includes a display, such as a touch screen display to allow user input to be registered and then steps are taken by the main controller (main processor).

In one exemplary embodiment, the main controller can be used to control the operation of the electrodes 5. In other words, select electrodes 5 can be operated (activated) at a given time using the main controller. Those electrodes 5 that are activated are supplied with ablative energy, while those electrodes 5 that are not activated are not supplied with ablative energy. As mentioned, the electrodes 5 can be wired to an electrical connector that is itself connected to a terminal (console) or the like (e.g., an outlet or plug thereof), thereby providing power to the electrodes 5.

Depending upon certain parameters, such as the location of the balloon catheter in the body, certain electrodes 5 can be activated and turned on, while certain electrodes 5 can be turned off and not activated. For example, if the balloon catheter and tubular structure are contacting certain tissue and such contact with tissue is being visualized by an endoscope inside the balloon, the user may want only those electrodes 5 that are in contact with tissue to receive ablative energy and therefore, based on endoscopic guidance or the like, the operator can strategically select which branches 4 and electrodes 5 that are to be activated.

The master controller can communicate with a display on which images and data can be displayed.

A touch screen or the like can be used to select the branches 4 and electrodes 5 that are to be activated (energized). For example, a graphic image of the elongate structure 1 and more specifically, a graphic image of the branches 4 and the electrodes 5, can be displayed to the operator and then the operator can select those branches 4/electrodes 5 to be activated. When a touch screen is used, the operator can simply highlight and select with a finger those branches 4/electrodes 5 that are to be activated. It will also be appreciated that AI based software can be used to determine and then recommend to the user which electrodes should be activated based on the determination that those electrodes are in contact with tissue.

PFA Catheter Mounted Basket

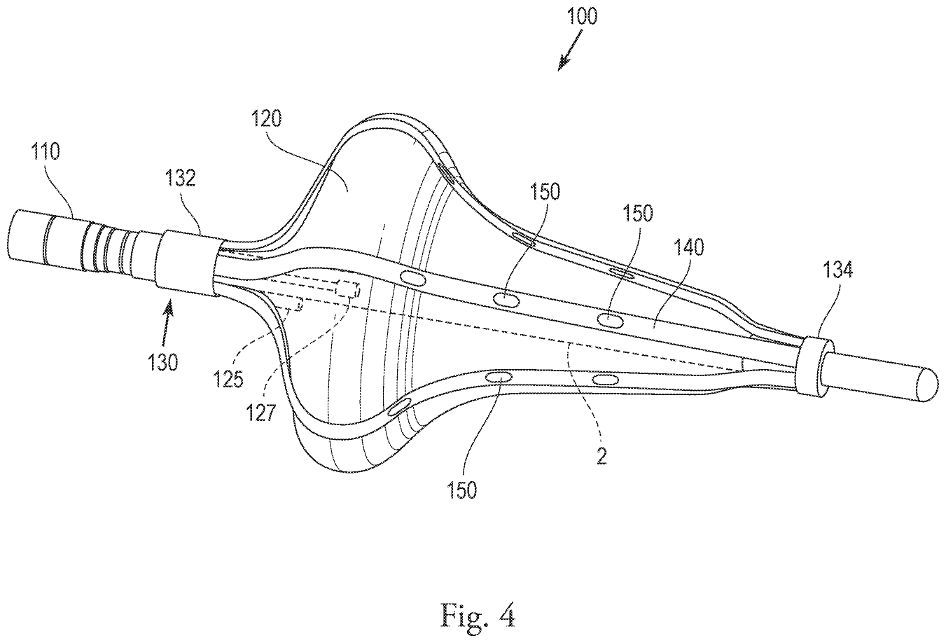

FIG. 4 illustrates a balloon catheter 100 that includes a main catheter shaft 110 that has a distal end. It will also be appreciated that the balloon catheter 100 typically includes more than one shaft and often includes an inner catheter shaft and an outer catheter shaft or can otherwise include multiple concentric tubular structures. An inflatable balloon 120 is included and is coupled to the main catheter shaft 110 with a distal end of the inflatable balloon 120 being proximate the distal end of the main catheter shaft 110 and the proximal end of the inflatable balloon 120 being spaced from the distal end. The inflatable balloon 120 thus surrounds the main catheter shaft 110.

FIG. 4 also shows an inner shaft 115 along with an endoscope 125. The endoscope 125 extends along the exterior of the inner shaft 115 and is typically located at one end of the balloon is forward looking in that it looks forward toward the other end of the balloon.

The inflatable balloon 120 is preferably a compliant balloon.

The inflatable balloon 120 also includes an endoscope 125 that is located within the compliant balloon. The endoscope allows the operator of the catheter to visualize the balloon surface and thereby aim the laser energy to those portions of the balloon surface which contact the atrial tissue it is desired to treat with the laser energy. Such a system is described in Melsky et al. (U.S. Pat. No. 9,421,066 (the '066 patent) and Melsky et al. (U.S. Pat. No. 9,033,961 (the '961 patent), each of which is incorporated by reference in its entirety. The endoscope is at a location that is proximal to the location at which the energy is delivered to the tissue to allow the user to view the delivery of the energy and the resultant tissue lesion(s). The endoscope can be one of the ones described herein and also one that is described in any one of the documents incorporated by reference herein.

In FIG. 4, an energy emitter 127 is illustrated; however, it will be appreciated that in the embodiment in which the electrode array is intended to remain in a position surrounding the inflatable balloon 120, then the energy emitter 127 can be eliminated or is present but never used. In the event that the electrode array can be displaced off of the balloon, then the energy emitter 127 can be used.

The endoscope 125 is forward-facing and is disposed adjacent one the catheter shafts, such as a central tubing typically formed of a transparent polymer material. As used herein, the term forward-facing refers to the view of the endoscope in a distal direction relative to the catheter body. Similarly, the term side-facing refers to the view of the endoscope in a direction that is radially outward from a side of the catheter body.

The endoscope 125 can be a fiber optic endoscope that is inserted through a lumen of the catheter and located within a proximal region of the inflatable balloon 120.

In another embodiment, the ablation catheter 100 includes first and second imaging devices for providing direct visualization of the region to be treated, with the first imaging device being fixed relative to the catheter body. The first and second imaging devices can be in the form of first and second imaging chip endoscopes. Details of the first and second imaging chip endoscopes are described in U.S. patent application Ser. No. 17/524,472, which is expressly incorporated herein by reference in its entirety.

The balloon catheter 100 includes an expandable basket 130 that surrounds the inflatable balloon 120 and is configured to expand upon expansion (inflation) of the inflatable balloon 120 and similarly, is configured to contract upon deflation and contraction of the inflatable balloon 120. The expandable basket 130 has a first collar (first ring) 132 at a first (proximal) end of the expandable basket 130 and a second collar (second ring) 134 at a second (distal) end of the expandable basket 130. The first and second collars 132, 134 have annular shapes and can thus have a continuous ring shape. The size of the two collars 132, 134 can be different from one another with the first collar 132 in the illustrated embodiment being larger than the second collar 134. The two collars 132, 134 are sized and configured to fixedly couple the expandable basket 130 to the main catheter shaft 110 (or one or more other catheter shafts) with the inflatable balloon 120 being located between the two collars 132, 134. The first collar 132 is thus preferably located proximal to the inflatable balloon 120, while the second collar 134 is located distal to the inflatable balloon 120.

The expandable basket 130 includes a plurality of splines 140 that are attached at one end to the first collar 132 and at the other end to the second collar 134. The plurality of splines 140 extend longitudinally along a length of the inflatable balloon 120. The plurality of splines 140 are circumferentially offset from one another with open spaces formed between adjacent splines 140. The splines 140 are constructed to expand and contract under action of the underlying inflatable balloon 120. In particular, when the inflatable balloon 120 expands under inflation, the splines 140 expand outwardly and conversely, when the inflatable balloon 120 contracts under deflation, the splines 140 contract inwardly. The splines 140 thus conform to the shape of the inflatable balloon 120.

Each spline 140 carries one or more electrodes 150. For example, each spline 140 can include a plurality of electrodes 150 that can be described as being an electrode array. In the illustrated embodiment, there are three electrodes 150 located along the length of the spline 140. The electrodes 150 are spaced longitudinally along the spline (in series). The electrodes 150 are thus spaced apart from one another a predefined set distance. The locations of the splines 140 along the spline 140 are selected so as to centrally position the electrodes 150 relative to the inflatable balloon 120 since when the inflatable balloon 120 is inflated, the electrodes 150 are, as discussed herein, for placement against the target tissue to be ablated using PFA technique.

The electrodes 150 that define the electrode can be the same electrode type or they can be different. For example, the shapes and sizes of the electrodes 150 can be the same as shown. The material of the expandable basket 130 is not elastic in that the splines do not stretch elastically in a longitudinal direction but can expand and contract with the underlying inflatable balloon 120. Thus, the longitudinal spacing between the electrodes 150 does not change when the expandable basket 130 moves between the expanded position and the retracted position. Instead, it is a fixed distance which is important and this information is used during the visualization and ablation process in order to form the desired lesion as discussed herein.

Compared to the embodiment of FIGS. 1-3, FIG. 4 shows a product in which the expandable basket 130 is fixed at least in one embodiment.

In yet another aspect of the present disclosure, the system can include electrode markers which mark the location of the electrodes 150 along the splines. In particular, the electrodes 150 are located on the outer surface of the splines 140 and the splines are typically formed of a non-transmissive material and therefore, the electrodes 150 are not visible in the live endoscope image. Since the splines 140 are typically formed of an opaque material, the electrodes 150 cannot be seen since the endoscope 125 only sees the inner surface of the splines 140. In order for the locations of the electrodes 150 to be determined during visualization (i.e., use of the endoscope 125), markers can be provided along the inner surface of the spline 140. Each marker is located on the inner surface of the spline 140 directly opposite the location of the electrode 150 to mark the location of the electrode 150. The markers are visually identifiable in the live endoscopic image and therefore can be in the form of visual indicia formed along the inner surface of the spline 140. For example, the visual indicia can be in the form of numbers and/or text indicia. In addition, the visual indicia is selected such that one electrode can be differentiated from another electrode. For example, each spline can be numbered, such as spline 1, and then each electrode 150 can be lettered, such as A, B, C, etc. Thus, in the illustrated embodiment, the most distal electrode of spline 1 can be identified by marker 1A, the middle electrode can be identified by marker 1B and the most proximal electrode can be identified by marker 1C. Similarly, for the adjacent spline 2, the markers can be 2A, 2B, and 2C. It will be appreciated that there are many different ways to visually identify one electrode on one spline from another electrode on another spline.

For example, color can be used to identify one spline 140 from the other ones. For example, the letters A, B, C or numbers 1, 2 and 3 can be in one color for one spline and another color for another spline. Symbols can also be used as the markers.

It will be appreciated that not all of the electrodes 150 are visible in the live endoscopic image since not all of the electrodes are in the desired contact with tissue at the target site and therefore it is important to understand which electrodes are visible in the live endoscopic image and in contact with tissue so that these electrodes can be actuated (activated).

The movement of the expandable basket 130 and the inflatable balloon 120 can vary depending on the embodiment. For example, in one embodiment, the expandable basket 130 and the inflatable balloon 120 can move together, while in another embodiment, the basket 130 can move independent from the balloon 120. For example, the basket 130 can be fixed in the rotational direction but can move in the axial (longitudinal direction) or in another embodiment it can be fixed.

The movement of the expandable basket 130 relative to the catheter body and the inflatable balloon 120 can be either an automated process as by using an electronic controller or it can be a manual process that occurs under action of the user. The controls permit the desired movements in rotational and/or longitudinal directions.

Delivery of energy and electrode selection: In one embodiment, energy is delivered to two or more electrodes 150 that are located along the same spline 140. In this embodiment, since the distance between the electrodes 150 on one spline 140 is fixed and does not change based on the expansion of the basket. This allows the PFA dosing to be selected since the distance between the electrodes 150 to be activated is known. In another embodiment, energy is delivered between two electrodes 150 that are not located along the same spline 140 but rather are located along adjacent splines 140. In this case, the distance between the splines 140 does change depending on the degree of basket expansion. For example, the greater the degree of basket expansion, the greater the distance between the splines 140 and thus, the greater the distance between the electrodes 150. When the electrode spacing remains fixed, there is a greater degree of dosing predictability.

In view of the visualization information and the location of and spacing between the electrodes that are to be actuated to cause lesion formation, the (PFA) dosing is selected. The correct (optimal) dose is one which provides good tissue isolation but does not adversely affect tissue quality.

In ablating tissue, certain select electrodes 150 are activated as opposed to activating all of the electrodes 150. Only those electrodes 150 that are in direct contact with tissue are activated to delivery energy and form the tissue lesion.

Depending upon the visualization information, the basket 130 may need to be moved axially and/or rotationally in order to perform the ablation. For example, if the electrode spacing is too great, energy is delivered to form a first lesion segment and then the basket may need to be moved relative to the balloon (axially and/or rotationally) to reposition the electrodes and deliver energy to form a second lesion segment that is combined with the first lesion segment to form a more complete lesion segment. Alternatively, circumferential electrode spacing can be inferred from the endoscopic view and the PFA dosing adjusted to compensate for differing electrode spacing.

The shape and size of the formed lesion segment will depend on which electrodes were actuated and their locations. For example, activation of two electrodes 150 located along the same spline 140 will result in a formed lesion that extends more longitudinally, while activation of two electrodes 150 located along adjacent splines results in a formed lesion that extends more in a circumferential direction.

Dual Transeptal/Secondary Catheter

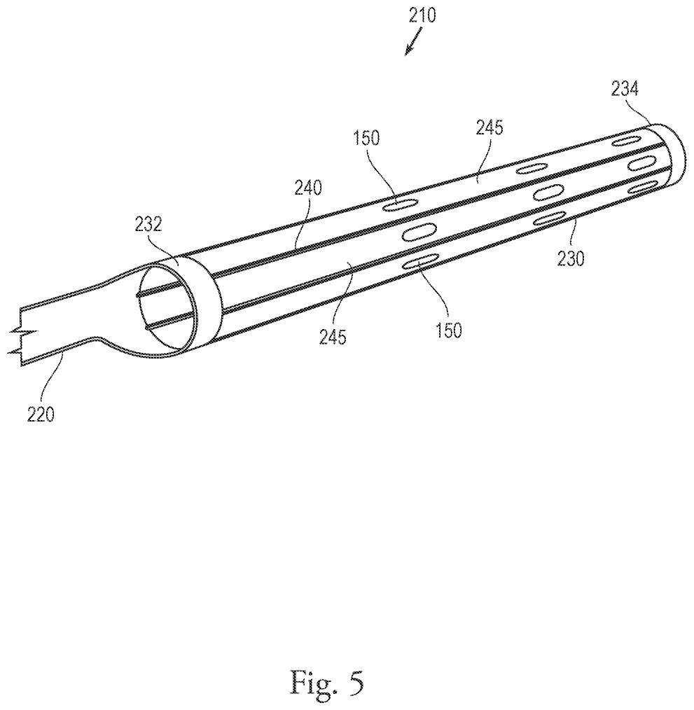

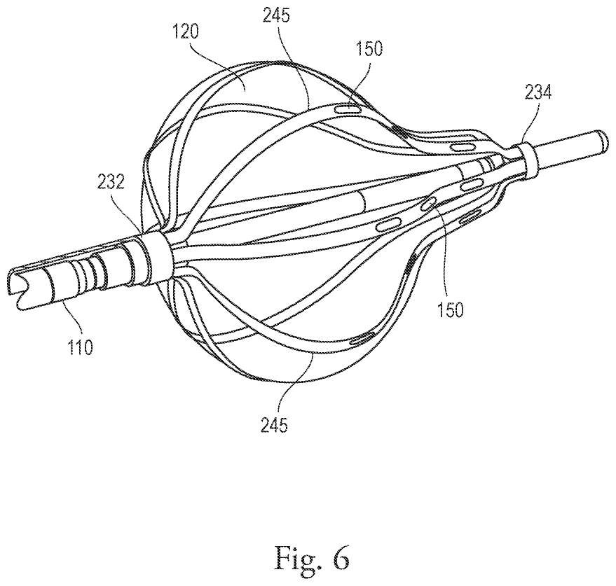

FIGS. 5 and 6 illustrate a balloon catheter 200 that is similar to the balloon catheter 100 with the exception that the balloon catheter 200 does not include the expandable basket 130. As a result, the reference numbers used in FIG. 4 are also used in FIGS. 5 and 6 for the parts that are in common to the two embodiments. The inflatable balloon is typically transparent and thus FIG. 6 shows the transparent nature of the balloon.

The balloon catheter 200 includes the main catheter shaft 110 that typically includes more than one shaft and often includes an inner catheter shaft and an outer catheter shaft or can otherwise include multiple concentric tubular structures as shown. The inflatable compliant balloon 120 is included and is coupled to the main catheter shaft 110 with a distal end of the inflatable balloon 120 being proximate the distal end of the main catheter shaft 110 and the proximal end of the inflatable balloon 120 being spaced from the distal end. The inflatable balloon 120 thus surrounds the main catheter shaft 110.

In this embodiment, there is a second catheter, namely, an electrode catheter 210 that is for use with the balloon catheter 200. The electrode catheter 210 comprises an elongated structure that has an open distal end and has a proximal region 220 and a distal electrode region 230. The proximal region 220 can comprise an elongated arcuate shaped body that is not completely circumferential in shape. Conversely, the distal electrode region 230 can be a completely circumferential structure. The distal electrode region 230 includes a proximal collar 232 at a proximal end of the distal electrode region 230 and a distal collar 234 at a distal end of the distal electrode region 230. Between the two collars 232, 234, the body of the distal electrode region 230 includes a plurality of longitudinal slits 240 that are spaced circumferentially about the body. These slits 240 define a plurality of longitudinal splines 245. The slits 240 do not extend into the areas of the two collars 232, 234. Much like the splines 140, the splines 245 carry one or more and preferably a plurality of the electrodes (e.g., electrodes 150) that are located along the outer surface (outer face) of the splines 245. Much like the previous embodiment, each spline 245 can carry a plurality of electrodes, such as three of more electrodes that are disposed in series and spaced apart from one another in the longitudinal direction of the spline 245.

Both ends of the distal electrode region 230 are open and thus, it represents an open ended tubular structure that, as described herein, is configured to receive the balloon catheter in its contracted (deflated) at rest state.

As in the previous embodiment, the splines 245 are not elastic and thus do not stretch but can expand in response to the expansion of the inflatable balloon 120. Therefore, the distance between the electrodes along the same spline 245 do not change based on whether the spline 245 is expanded or retracted. However, as in the previous embodiment, the distance between two electrodes on two different splines 245 does change based on the degree of expansion.

The balloon catheter is inserted into and through the hollow interior (inner lumen) of the electrode catheter 210 such that the splines 245 surround the inflatable balloon 130. As the balloon inflates, the splines 245 expand radially outward and separate from one another.

As in the other embodiment, the splines 245 can collapse as by retracting the splines 245 inside a main (outer) catheter shaft.

Visualization is used in this embodiment also to determine which electrodes are in contact with the tissue and also the visualization can guide the user in terms of making any adjustments with the balloon catheter and/or the electrode catheter in order to form a complete continuous lesion.

Retractable Tines Electrode Array

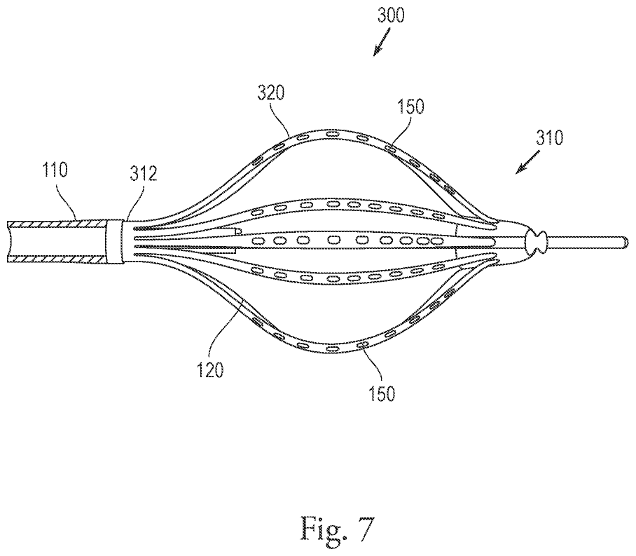

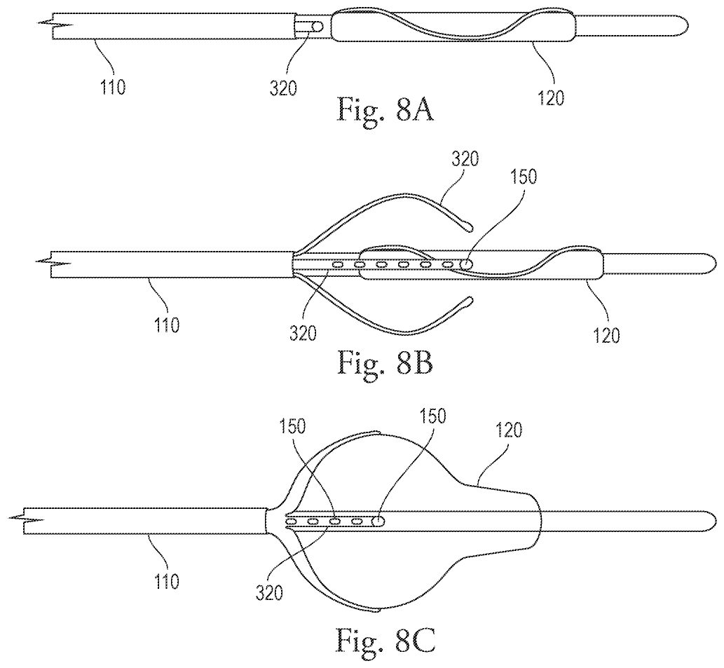

FIGS. 7 and 8A-C illustrate a balloon catheter 300 that is similar to the balloon catheter 100 with the exception that the balloon catheter 300 does not include the expandable basket 130. As a result, the reference numbers used in FIG. 4 are also used in FIGS. 7 and 8A-C for the parts that are in common to the two embodiments.

The balloon catheter 300 includes the main catheter shaft 110 that typically includes more than one shaft and often includes an inner catheter shaft and an outer catheter shaft or can otherwise include multiple concentric tubular structures as shown. The inflatable balloon 120 is included and is coupled to the main catheter shaft 110 and/or an additional shaft with a distal end of the inflatable balloon 120 being proximate the distal end of the main catheter shaft 110 and the proximal end of the inflatable balloon 120 being spaced from the distal end. The inflatable balloon 120 thus surrounds the main catheter shaft 110.

The balloon catheter 300 further includes a retractable electrode sheath 310 that is configured to retract within the main catheter shaft 110 or another shaft of the catheter. Thus, as described herein, the retractable electrode sheath 310 is designed to move longitudinally along the main catheter shaft 110 and more particularly, the retractable electrode sheath 310 can travel within the main catheter shaft 110 to allow the retractable electrode sheath 310 to move between a fully retracted position and a fully extended position. In the fully retracted position, at least a substantial length of the retractable electrode sheath 310 is contained within the main catheter shaft 110 and in the fully extended position, a substantial length of the retractable electrode sheath 310 is disposed outside of the main catheter shaft 110 and surrounds the inflatable balloon 120 as described herein. As shown, in the fully extended position, the tines 320 can extend at least 75% of the length of the balloon 130 and can extend over 90% of the length of the balloon. In another embodiment, the tines 320 extend at least 50% of the length of the balloon 130 (e.g., they extend at least to the widest part of the inflated balloon 130).

The retractable electrode sheath 310 includes a proximal collar 312 that can be a continuous cylindrical structure and a plurality of expandable tines 320 that are integral at their proximal ends to the proximal collar 312. The tines 320 are cantilevered structures in that a distal end of each tine 320 is a free end and is not attached to another structure. The tines 320 are spaced apart and extend circumferentially around the balloon 130 when in the fully extended positions.

As in the other embodiments, the tines 320 are not elastic and do not stretch in any way; however, the tines 320 are able to expand (radially) outward as the inflatable balloon 130 inflates and similarly, when the inflatable balloon 130 deflates, the tines 320 can contract. The tines 320 thus can conform to the compliant balloon 130.

To cause retraction and full collapse of the tines 320, the retractable electrode sheath 310 is pulled back in the proximal direction and as the retractable electrode sheath 310 enters into the main catheter shaft 110, the presence of the main catheter shaft 110 in surrounding manner, applies an inward force to the tines 320 that collapses them and allows them to travel within the main catheter shaft 110 and retract away from the balloon 130.

As shown in the figures, each tine 320 includes one or more electrodes 150 and preferably a plurality of electrodes 150 that are spaced along the tine 320. The electrodes 150 are disposed in series along the length of the tine 320. The electrodes 150 along the tine 320 can be of the same type (e.g., same shape and size, etc.) or different type electrodes can be used in another embodiment.

As in the other embodiments, visualization (e.g., the endoscope) is used to determine which electrodes 150 are in contact with the tissue and those select electrodes can then be activated (actuated) to form the lesion. The user interface allows for the identification and powering of those electrodes 150 that are in contact with the tissue. As mentioned previously, the operating software can be programmed so that based on the distance between the activated electrodes 150, the proper dosing amount can be calculated and the requisite energy can be delivered to the electrodes 150.

As in all embodiments, it is desirable to limit the activation of electrodes to only those that are required to form the lesion (segment).

FIG. 8A shows the inflatable balloon 130 in a deflated state and the tines 320 are fully retracted and are located substantially within the main catheter shaft 110 (e.g., only the tips of the tines 320 protrude outside of the main catheter shaft 110).

FIG. 8B shows the inflatable balloon 130 still in its deflated state but the tines 320 have been deployed. As mentioned, the degree of coverage of the tines 320 relative to the balloon 130 can vary.

FIG. 8C shows the balloon 130 inflated and this results in the expansion of the deployed tines 320. In this figures, the tines 320 are shown extended about 50% the length of the balloon 130; however, this is merely exemplary in nature and it will be understood that it can extend along more or less of the balloon length.

The embodiment of FIGS. 7 and 8A-C thus consists of semi-rigid retractable tines 320 with one or more electrodes 150 along the outer surface of each tine 320, that are housed within the catheter (main catheter shaft 110) and deployed before inflating the balloon 130 (by sliding the retractable electrode sheath 310 distally using a controller or the like (manual or motorized). When the balloon 130 is inflated, the electrodes 150 are pressed against the inner surface of the vessel to achieve tissue contact. As with the other embodiments, the endoscope in this embodiment within the balloon 130 to allow for confirmation of tissue contact and electrode spacing under direct visualization, once tissue contact and desired electrode spacing is confirmed, energy is applied to the desired (selected) electrodes 150 to create the lesion. This embodiment can incorporate as few as four deployable tines 320, but a larger number of tines 320 is likely to provide the user with the ideal number of electrodes 150 and electrode spacing for effective treatment.

In this embodiment, as in the other embodiments, markers can be provided along the inner surface of the tine 320 to identify the location of the electrodes 150 along the tine 320 under visualization. This allows the user to determine which electrodes 150 are in contact with the tissue and then instruct the energy delivery module to delivery energy to those selected electrodes 150. In addition, in one embodiment, the system can include image recognition software that analyses the live image feed from the endoscope and identifies the electrode markers that are present. For example, if the markers, such as A1 and A2, are present, then the image recognition module will identify these electrodes and provide the user with the option to confirm that the electrodes that correspond to markers A1 and A2 should be activated and energy delivered to the user.

This image recognition functionality can be implemented in any of the other embodiments described herein in which the electrode markers are present to provide the user with a suggested electrode activation plan.

Balloon with PFA Braided Wire Mesh Electrode Array

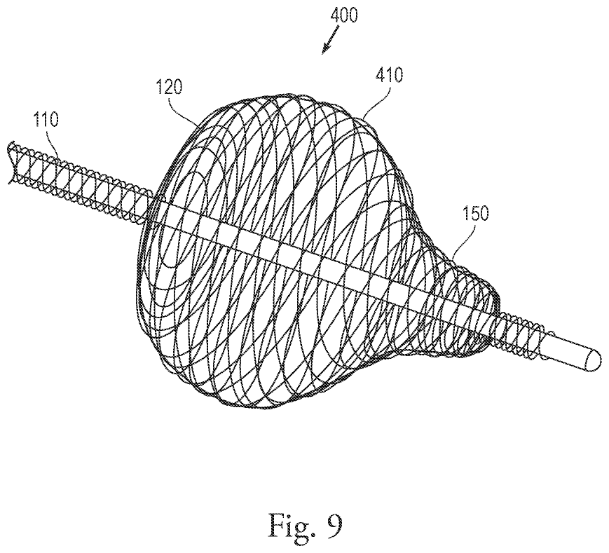

FIG. 9 illustrates a balloon catheter 400 that is similar to the other balloon catheters described herein. As a result, the reference numbers used in FIG. 4 are also used in FIG. 9 for the parts that are in common to the two embodiments.

The balloon catheter 400 includes the main catheter shaft 110 that typically includes more than one shaft and often includes an inner catheter shaft and an outer catheter shaft or can otherwise include multiple concentric tubular structures. The inflatable balloon 120 is included and is coupled to the main catheter shaft 110 with a distal end of the inflatable balloon 120 being proximate the distal end of the main catheter shaft 110 and the proximal end of the inflatable balloon 120 being spaced from the distal end. The inflatable balloon 120 thus surrounds the main catheter shaft 110.

The balloon catheter 400 includes a wire braid 410 that is disposed over the inflatable balloon 120 and is configured to expand radially as the inflatable balloon 120 is inflated. The wire braid 410 can comprise a mesh wire braid as shown. This wire mesh can be used as a support structure for an electrode array formed of electrodes 150 and can be formed of an insulating material. The electrodes 150 are disposed along the outer surface of the wire braid 410 and the coverage of the electrodes 150 can be uniform of non-uniform. In the non-uniform embodiment, the electrodes 150 can be more concentrated in one or more regions of the wire braid 410. For example, the electrodes 150 can be primarily located at the center region of the wire braid 410 where tissue contact is more likely.

In addition, the spacing between electrodes can be the same along the entire electrode array or the spacing can be different in one or more regions of the wire braid 410. For example, the spacing can be closer together in a central region of the wire braid 410.

As in the other embodiments, the electrodes 150 are connected to the energy source using conventional electric traces or wires (conductive paths) that are associated with and/or incorporated into the wire braid.

Alternatively, the wire braid 410 (support structure) itself can serve as and define the electrode array by incorporating an insulating coating on a conductive (metal) braid wires that is stripped off at desired locations for energy delivery by defining discrete electrodes in those area where the coating is removed. The wire braid 410 would be operatively connected to the energy source and electric current (energy) is delivered across the wire braid 410 with the areas in which the insulating coating is removed defining the electrodes that define the electrode array.

The wire mesh braid can be formed of separate discrete insulated wires to define discrete pathways along which the electrodes are present. By defining discrete electrode pathways, discrete regions of the wire mesh braid can be activated without activation of the other regions to allow for activation of those electrodes or that electrode region that is in contact with the tissue.

As shown, the wire braid 410 can extend beyond the inflatable balloon 130 in that one end of the wire braid 410 extends proximal to the inflatable balloon 130 and the other end of the wire braid 410 extends distal to the inflatable balloon 130.

As with the other embodiments, this embodiment once again uses an endoscope inside the balloon 130 in order to confirm electrode placement and tissue contact. The number of electrodes 150 in the array may vary along with the number of braid wires in order to achieve the most clinically effective energy delivery, and the user may be able to select or deselect a number of electrodes in order to customize the treatment zone.

Balloon with Embedded Electrode Array

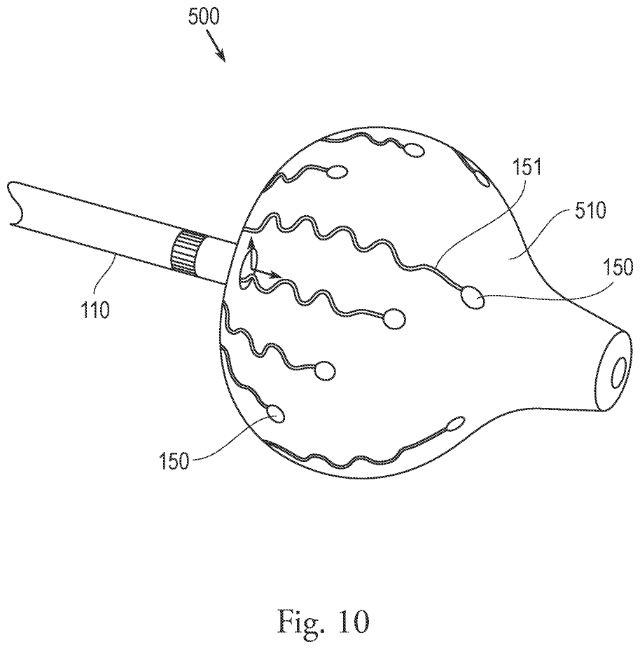

FIG. 10 illustrates a balloon catheter 500 that includes the main catheter shaft 110 along with an inflatable balloon 510 that is coupled to and extends along the main catheter shaft 110 as in the other embodiments. The distal end of the inflatable balloon 510 is coupled to a distal end of the main catheter shaft 110 and a proximal end of the inflatable balloon 510 is coupled to the main catheter shaft 110 at a location spaced from the distal end of the main catheter shaft 110.

The inflatable balloon 510 is a compliant balloon in which the electrodes 150 are integral. The balloon 510 itself includes electrodes 150 and flexible wire traces 151 embedded in the balloon material.

In this embodiment, the electrodes 150 can be disposed in and made integral to the balloon 510 as part of the molding process of the balloon 510. The electrodes 150 are spaced across the balloon 510 in a desired pattern. For example, the electrodes 150 are located circumferentially around the balloon 510. Alternatively, instead of being positioned and attached to the balloon material during the manufacture process, the electrodes 150 can be attached to the balloon 510 after the manufacture process. In particular, the electrodes 150 can be attached to the outer surface of the balloon 510 with the traces 151 also being attached to the outer surface of the balloon 510. Any number of conventional techniques can be used to attach these elements to the outside of the balloon 510 such as use of adhesive, bonding agents, etc.

The electrodes 150 are formed so that an outer surface of each electrode 150 is exposed along the surface of the balloon 150 for placement in contact with the tissue. Each flexible trace 151 is formed in a zig-zag pattern which is purposeful in order to permit the flexible traces 151 to move with the compliant balloon during inflation/deflation and during placement against the tissue. In other words, this zig-zag pattern accommodates the flexible traces 151 during the expansion and contraction of the balloon and prevents damage to the trace(s). Each flexible trace 151 is operatively coupled to the energy source so that energy can be delivered to select ones of the electrodes 150.

As with the other embodiments, this embodiment once again uses an endoscope inside the balloon 510 in order to confirm electrode placement and tissue contact. Once the user determines which electrodes 150 are in contact with the tissue, the user can then select these electrodes for activation.

In addition, electrode markers can be provided as in the other embodiments that are visible on the inside of the balloon 510 to the endoscope to allow the user or to allow image recognition software to determine which electrodes are clearly visible in the field of view of the endoscope. Based on this information, energy is delivered to those select electrodes 150 for forming the lesion. The user interface can be configured to easily allow the user the ability to select which electrodes to deliver energy to as by presenting the user with a touch screen with an electrode map and/or having the image recognition software prepopulate the screen with a proposed electrode activation map indicating which electrodes are visible in the endoscope and in contact with tissue.

Balloon with Micropores and Inner Electrode Array

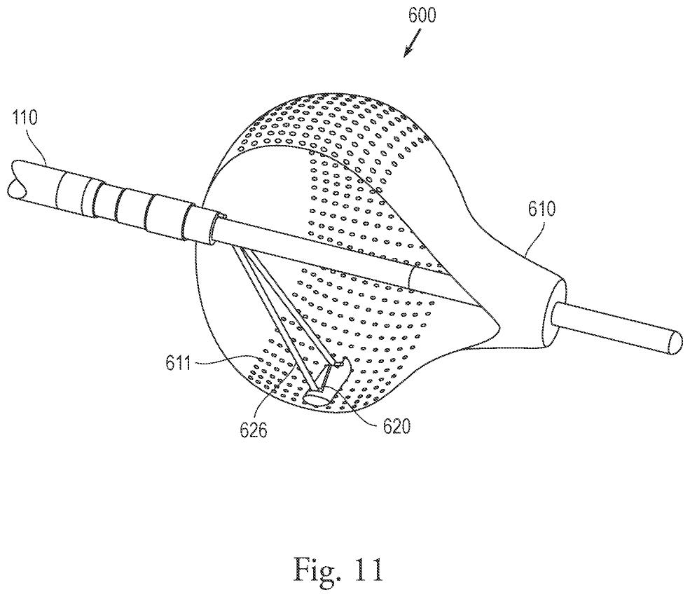

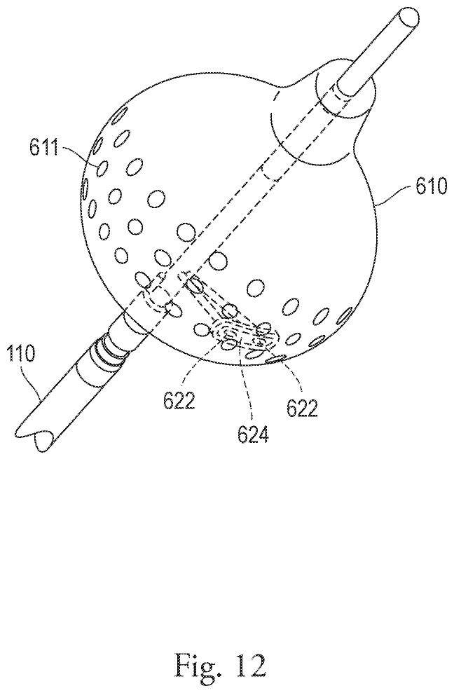

FIGS. 11 and 12 illustrate a balloon catheter 600 that includes the main catheter shaft 110 along with an inflatable compliant balloon 610 that is coupled to and extends along the main catheter shaft 110. An outer catheter body or sleeve 115 is also present and as mentioned, the catheter 600 can include other shafts, such as outer and inner catheter shafts, etc. The distal end of the inflatable balloon 610 is coupled to a distal end of the main catheter shaft 110 and a proximal end of the balloon 610 is coupled to the main catheter shaft 110 at a location that is spaced from the distal end.

As with the other embodiment, an endoscope is provided inside of the balloon 610 and can be coupled to the main catheter shaft 110. The endoscope is forward looking and allows view of the transparent balloon 610 and its contact with surrounding tissue.

In accordance with this embodiment, at least a portion of the balloon 610 has micropores 611 formed therein. The micropores 611 are preferably formed in one or more regions of the balloon 610 in which energy is to be delivered to the tissue. In the illustrated embodiment, the proximal and distal ends of the balloon 610 are devoid of micropores 611, while the center region includes the micropores 611 since it is this center region that contacts the tissue during use.

For ease of simplicity, the micropores 611 in FIG. 12 are shown as having greater dimensions than the micropores in FIG. 11; however, it will be understood that the micropores in FIGS. 11 and 12 can be the same size and the same number. However, FIG. 12 does convey that the micropores 611 can be formed to have different sizes and even different shapes.

The micropores 611 can have uniform constructions (i.e., same size and shape) or there can be two or more types of micropores 611. The micropores 611 can be formed in a uniform pattern as shown or can be formed in a non-uniform pattern. For example, as illustrated, the micropores 611 can be formed in a grid that extends circumferentially around the entire balloon 610.

The balloon catheter 600 also includes an electrode carrier 620 that is disposed within the balloon and can, in at least one embodiment, move within the balloon 610 (i.e., move rotationally within the balloon 610 and/or move longitudinally within the balloon 610). The electrode carrier 620 includes one or more electrodes 622 that are contained in a housing (hood) 624. In the illustrated embodiment, there is a pair of electrodes 622 in the housing 624 (however, it is possible to use a single electrode in the hood, with the hood rotating within the inside of the porous balloon). The housing 624 serves to contain and direct the energy of the electrodes 622. The electrodes 622 are placed in close proximity to the balloon itself, and the housing itself is placed in direct contact with the balloon's inner surface. The hood 624 can optimize the fraction of ablative energy delivered to tissue; however, the hood 624 can be eliminated and is not necessary.

The electrode array 622 is thus contained in the housing 624 which also serves to encapsulate a conductive liquid media, such as saline (e.g., normal saline or hypertonic saline) that allows for energy flow directly into the tissue via the micropores 611. In other words, the conductive liquid media can be delivered to the housing 624 as by use of one or more conduits 626 that open up into the inside of the housing 624. When the electrodes 622 are activated, energy is produced by the electrodes (e.g., between the electrodes) and since the electrodes 622 are bathed in the conductive liquid media, the energy serves to heat the conductive liquid media. The presence of the micropores 611 allows the heated conductive liquid media to weep through the micropores 611 to the tissue, which in combination with the energy from the electrodes 622 being conducted across the balloon material results in a target lesion being formed. In particular, a lesion segment is formed. To form a complete lesion, the electrode carrier 620 can be rotated and/or moved along the inner surface of the balloon. The electrode carrier 620 is held in contact with the inner surface of the balloon 610 by means of a mechanical adjustment controlled by the user, or a secondary balloon that can be inflated or deflated by the user to adjust the electrode contact pressure.

The combination of the electrode array and the conductive liquid media defines an electroconductive pathway used to form the lesion segment. It will be appreciated that the inflation media to control inflation or deflation of the balloon 610 can be the same or different than the conductive liquid media delivered to the inside of the housing 624.

In yet another embodiment, the balloon 610 does not include micropores 611 but instead is formed of a conductive balloon material (e.g., balloon material doped with carbon nanotubes). In this alternative embodiment, the housing (hood) can also be eliminated or it can be maintained. A non-conductive fluid can thus be used inside the balloon. The electrode array (or single electrode) is still disposed inside of the balloon 610 and is movable therein as by being able to freely rotate within the balloon and/or move longitudinally. Energy delivered to the electrode array is thus transferred to a local region of the conductive balloon that is in close proximity to the electrode array to form the lesion. In other words, the electrode array faces a localized area of the balloon and energy that is delivered to the electrode array is conducted to this localized area of the balloon to form the lesion.

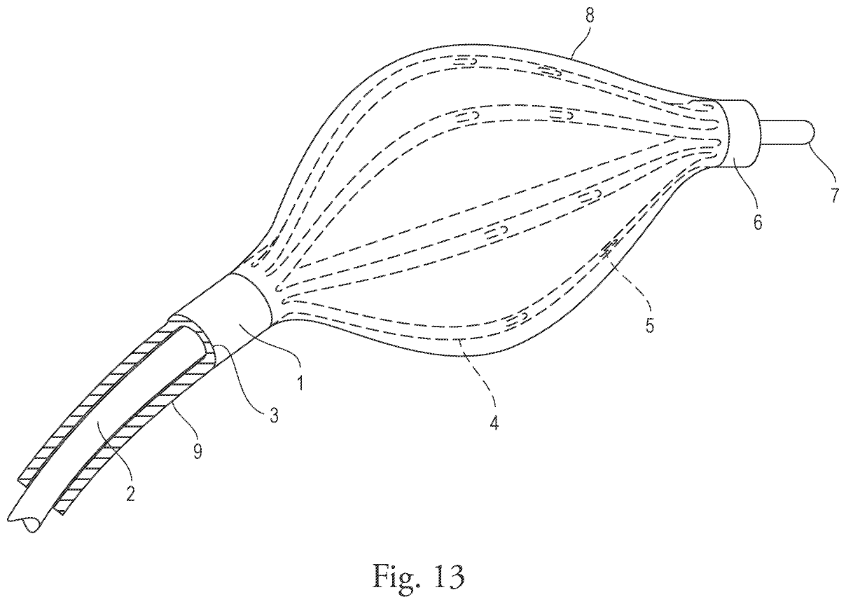

Now referring to FIG. 13, in yet another embodiment, a porous balloon catheter 700 is shown. The porous balloon catheter 700 is similar the balloon catheter 600 and therefore, like elements are numbered alike. The balloon thus includes micropores 611. Instead of the electrode carrier 620, the balloon catheter 700 includes an elongate structure 710 that can be similar to the elongate structure 1 of FIG. 1 with several notable differences being that the elongate structure 710 is located inside the balloon as opposed to being located outside the balloon as in FIG. 1. The elongate structure 710 comprises a first tubular shaped portion 712 and a second tubular shaped portion 714 that surround the catheter shaft. The elongate structure 710 multifurcates into two or more but preferably six or more branches 720, with each branch 720 containing one or more electrodes 715 on their outward facing surface. The elongate structure 710 can be made of an elastic material pre-shaped into a geometry that allows it to expand and remain in contact with the balloon inner surface as the balloon is inflated. The elongate structure 710 would be collapsed by the balloon when the balloon is deflated by removing liquid from the balloon under vacuum. In other words, as the balloon is inflated, the elongate structure is constructed to automatically and naturally expand and similarly, it contracts due to the contraction of the balloon. This can naturally occur due to the memory characteristics of the elongate structure 710. The electrodes 715 that are on the outer surface of the elongate structure 710 are thus in contact with the inner surface of the porous balloon. As in the other embodiment, the balloon contains conductive fluid that passes through the micropores. Thus, energy from the electrodes 715 is conducted across the balloon itself and/or the conductive fluid within the balloon passes through the micropores to the target tissue.

It will be appreciated that in all embodiments, the electrodes are connected to a controllable energy source using conventional techniques, including electric leads, wires, conductive pathways, etc. The energy source can be controlled using traditional controls such as a master controller that can be a part of a console at which the user enters input and can control and select different operating parameters such as dosing information (dose power (wattage), etc.

Those embodiments that incorporate an electrode array are particularly suited for delivery of electroporative ablation energy (PFA).

Additional details concerning certain embodiments of the present disclosure are as follows.

A device for the alteration of tissue for the purpose of changing, amongst other things, the conducting properties of the tissue to achieve a desired result.

An external sheath that is positioned over the existing catheter system.

Consisting of three distinct portions, a location collar of a hard material at the most distal end, a balloon expandible section of softer, more pliable material (or alternative arrangement) located in the vicinity of the primary balloon and an overcoat on the body of the catheter continuing until near the proximal end.

Electrodes may be placed on the hard collar section for measurement of distal electrical activity or may be employed in the delivery of energy.

Electrodes are primarily placed on the balloon expandible section, for the delivery of energy to achieve the alteration of the properties of the target tissue, in a variety of configurations (another section)

The overcoat of the body incorporates the conductors for the distal measurement and energy delivery and are terminated in the vicinity of the control for the rotation of the other energy delivery source.

The electrodes on the collar may be in a variety of configurations, including square electrodes in a 2, 4, or 6 style equally spaced around the measuring area on the collar.

The balloon expandible area electrodes are intended to be the primary energy delivery (therapeutic) of the device. The most likely embodiment would be 16 electrode arrangement, equally spaced, positioned proximally to the primary treatment area, allowing the balloon to be deflated slightly to allow the electrode array to be extended distally into the area to be treated, perhaps, but not necessarily in an arc similar in location to where the primary energy was or will be delivered. The area will be aligned so that at the inflation pressure designated for “PFA” therapy, the electrodes will be equally spaced and are separate, so that they may be accessed individually or in a variety of groupings.

The overcoat of the catheter will have the conducting means for all of the sensing and energy delivery electrodes (some or all of them serve dual purposes) so as not to provide any, or at least a minimal amount, of impingement on the flexure or rotation of the primary catheter. This may be a spiral routing with the ability to use a variety of spiral pitches.

The balloon expandible section of the device may be a complete sheath consisting of a very elastic material with the electrodes on the surface or may be more rigid with sections of the device removed so that the electrodes are placed into the desired area by displacement of the structure.

It is to be understood that like numerals in the drawings represent like elements through the several figures, and that not all components and/or steps described and illustrated with reference to the figures are required for all embodiments or arrangements.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising”, when used in this specification, specify the presence of stated features, integers, steps, operations, elements, and/or components, but do not preclude the presence or addition of one or more other features, integers, steps, operations, elements, components, and/or groups thereof.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

The subject matter described above is provided by way of illustration only and should not be construed as limiting. Various modifications and changes can be made to the subject matter described herein without following the example embodiments and applications illustrated and described, and without departing from the true spirit and scope of the present invention, which is set forth in the following claims.

What is claimed is:

1. A method for ablating target tissue comprising the steps of:
delivering an ablation balloon catheter to the target tissue, wherein the ablation balloon catheter includes a compliant balloon, a visualization device; and an electrode array that is visible to the visualization device, each electrode being configured to deliver ablation energy, wherein the electrode array is independently movable relative to the compliant balloon;
isolating the target tissue such that at least one electrode of the electrode array is in contact with the target tissue; and
delivering the ablation energy to those electrodes of the electrode array that are confirmed, using the visualization device, to be in contact with target tissue.

2. The method of claim 1, wherein the visualization device comprises an endoscope that is disposed inside the compliant balloon.

3. The method of claim 1, wherein the visualization device is at a fixed location relative to a catheter shaft of the balloon catheter.

4. The method of claim 1, wherein the ablative energy is selected from the group consisting of: RF energy, laser, electroporative, and microwave.

5. The method of claim 1, wherein the ablation balloon catheter includes a longitudinally displaceable sleeve that is configured to move longitudinally along the balloon ablation catheter and over the compliant balloon, wherein the longitudinally displaceable sleeve has a proximal end portion, an opposing distal end portion and a plurality of branches that are connected to both the proximal end portion and the distal end portion, wherein the electrode array is disposed along an exterior of one or more branches of the plurality of branches.

6. The method of claim 5, wherein the ablation balloon catheter includes a main catheter shaft and the longitudinally displaceable sleeve is retractable within the main catheter to permit movement between a retracted position and an extended deployed position.

7. The method of claim 5, wherein the proximal end portion of the longitudinally displaceable sleeve comprises a first tubular structure and the distal end portion of the longitudinally displaceable sleeve comprises a second tubular structure and first ends of plurality of branches are connected to the first tubular structure and second ends of the plurality of branches are connected to the second tubular structure.

8. The method of claim 7, wherein a plurality of longitudinal slits are formed between the plurality of branches, thereby permitting radial expansion of the plurality of branches under inflation of the compliant balloon.

9. The method of claim 1, further including an electrode catheter separate from the ablation balloon catheter and being configured such that the ablation balloon catheter can pass completely through an inner lumen of the electrode catheter to allow the electrode catheter to be positioned along an exterior of the compliant balloon, the electrode catheter comprises an elongated structure that has an open distal end, a proximal region, and a distal electrode region that has a plurality of longitudinally splines separates from one another by a plurality of longitudinal slits, the electrode array being disposed along an exterior of one or more splines of the plurality of longitudinal splines.

10. The method of claim 1, wherein the ablation balloon catheter includes a longitudinally displaceable sleeve that is configured to move longitudinally along the balloon ablation catheter over the compliant balloon, wherein the longitudinally displaceable sleeve has a proximal end portion and a plurality of branches that are connected only to the proximal end portion at first ends of the plurality of branches, with opposite second ends of the plurality of branches being free ends, wherein the electrode array are disposed along an exterior of one or more branches of the plurality of branches.

11. The method of claim 1, wherein the compliant balloon comprises a porous balloon defined by micropores and the electrode array is part of an electrode carrier movably disposed within the porous balloon.

12. The method of claim 11, wherein the micropores are circumferentially formed about a first region of the porous balloon in which the ablative energy is to be delivered.

13. The method of claim 12, wherein a density of the micropores are greatest in the first region compared to second and third regions that are on either side of the first region and define proximal and distal regions, respectively, of the porous balloon.

14. The method of claim 11, the electrode carrier is free to rotate about a catheter shaft within the porous balloon.

15. The method of claim 11, wherein the electrode carrier is further configured to move axially and longitudinally along the catheter shaft.

16. The method of claim 11, wherein the electrode carrier includes a hood that surrounds the electrode array that comprises at least two electrodes within an interior of the hood, the electrode carrier including a fluid conduit that is in fluid communication with the interior of the hood for delivering a conductive fluid to the interior.

17. The method of claim 16, wherein the conductive fluid comprises a saline solution.

18. The method of claim 11, wherein the electrode carrier is configured to move in a radial direction relative to the catheter shaft to allow the electrode carrier to be placed in direct contact with or in close proximity to an inner surface of the porous balloon.

19. The method of claim 18, wherein the electrode carrier is mechanically coupled to the catheter shaft to allow the electrode carrier to move in the radial direction.

20. The method of claim 1, wherein the compliant balloon comprises a porous balloon defined by micropores and the electrode array is part of an electrode carrier movably disposed within the porous balloon.

21. The method of claim 1, wherein the ablation balloon catheter includes an electrode sleeve that is disposed within an interior of the porous balloon, wherein the electrode sleeve has a proximal end portion, an opposing distal end portion and a plurality of branches that are connected to both the proximal end portion and the distal end portion, wherein the electrode array is disposed along an exterior of one or more branches of the plurality of branches and is configured for placement against an inner surface of the porous balloon.

22. The method of claim 1, wherein the electrode array is disposed along an exterior of an electrode carrier that further includes electrode markers formed along an inner surface of the electrode carrier to mark locations of the electrodes of the electrode array, the electrode markers being visible to the visualization device.

23. The method of claim 5, wherein the ablation balloon catheter includes a movable energy emitter that is disposed within the compliant balloon, the movable energy emitter being configured to deliver a first type of energy through the compliant balloon to the target tissue, the method further including the steps of:
inflating the compliant balloon with the longitudinally displaceable sleeve disposed proximal to the compliant balloon such that the electrode array is not located over the compliant balloon;
delivering energy to the target tissue using the movable energy emitter;
deflating the compliant balloon;
moving the longitudinally displaceable sleeve over the compliant balloon such that the electrode array surrounds the compliant balloon;
inflating the compliant balloon; and
energizing at least some electrodes of the electrode array that in contact with the target tissue.

24. An ablation catheter system comprising:
a balloon ablation catheter including an inflatable balloon coupled to a shaft; and
a longitudinally displaceable sleeve that is configured to move longitudinally along the balloon ablation catheter, wherein the longitudinally displaceable sleeve having a proximal end portion, an opposing distal end portion and a plurality of branches that are connected to both the proximal end portion and the distal end portion, wherein the longitudinally displaceable catheter moves between a first position in which the longitudinally displaceable sleeve is located at a more proximal position relative to the inflatable balloon and a second position in which the longitudinally displaceable sleeve is disposed in a more distal position relative to the inflatable balloon,
wherein at least one branch includes at least one electrode located along an outer surface thereof;
wherein the plurality of branches are configured to deploy and radially expand under inflation of the inflatable balloon.

25. The ablation catheter system of claim 24, wherein the first proximal position is such that the longitudinally displaceable sleeve is located proximal of the balloon and the second more distal position is such that the longitudinally displaceable sleeve covers at least 50% of the inflatable balloon.

26. The ablation catheter system of claim 24, wherein the proximal end portion of the longitudinally displaceable sleeve comprises a first tubular structure and the distal end portion of the longitudinally displaceable sleeve comprises a second tubular structure and first ends of plurality of branches are connected to the first tubular structure and second ends of the plurality of branches are connected to the second tubular structure.

27. The ablation catheter system of claim 24, wherein the proximal end portion, the plurality of branches and the distal end portion are formed as a single integral part.

28. The ablation catheter system of claim 24, wherein a plurality of longitudinal slits are formed between the plurality of branches, thereby permitting radial expansion of the plurality of branches under inflation of the inflatable balloon.

29. The ablation catheter system of claim 28, wherein a width of each longitudinal slit is different than a width of each branch.

30. The ablation catheter system of claim 24, wherein the longitudinally displaceable sleeve is formed of a material selected from the group consisting of: a polyimide film, a polyester film; and a urethane film.

31. The ablation catheter system of claim 24, wherein the first position is a retracted position and the second position is an extended position in which the plurality of branches are disposed over the inflatable balloon.

32. The ablation catheter system of claim 24, wherein in the first position, each of the first tubular structure, the plurality of branches and the second tubular structure is located proximal to the inflatable balloon.

33. The ablation catheter system of claim 24, wherein in the second position, the second tubular structure is disposed over a distal tip of the shaft at a location distal to the inflatable balloon and the first tubular structure is disposed over the shaft at a location proximal to the inflatable balloon.

34. The ablation catheter system of claim 24, wherein a length of the plurality of branches is greater than a length of each of the first tubular structure and the second tubular structure.

35. The ablation catheter system of claim 34, wherein the length of the first tubular structure is greater than the length of the second tubular structure.

36. The ablation catheter system of claim 24, wherein each of the plurality of branches includes at least one electrode.

37. The ablation catheter system of claim 36, wherein each of the plurality of branches includes two or more electrodes.

38. The ablation catheter system of claim 37, wherein the two or more electrodes for each branch are longitudinally spaced apart.

39. The ablation catheter system of claim 24, further including a main controller that is operatively connected to each electrode to permit control over each electrode.

40. The ablation catheter system of claim 39, wherein in a first operating mode, all of the electrodes are activated by the main controller and wherein in a second operating mode, less than all of the electrodes are activated.

41. The ablation catheter system of claim 24, wherein the plurality of branches are circumferentially arranged.

42. The ablation catheter system of claim 24, wherein each electrode is connected to an insulated conductor wire imbedded in a body of the longitudinally displaceable sleeve.

43. The ablation catheter system of claim 24, wherein each branch is formed of a material that permits the branch to lengthen in a longitudinal direction when a radially outward force is applied by inflation of the inflatable balloon.

44. The ablation catheter system of claim 24, further including an endoscope and the longitudinally displaceable sleeve includes a plurality of electrode markers formed along an inner surface of the longitudinally displaceable sleeve, the at least one electrode comprising a plurality of electrodes disposed along an outer surface of the longitudinally displaceable sleeve and the plurality of electrode markers being formed opposite the plurality of electrodes to identify locations of the plurality of electrodes, the plurality of electrode markers being visible to the endoscope.

45. The ablation catheter system of claim 44, further including a display on which an image of the longitudinally displaceable sleeve is displayed and wherein a graphical user interface is provided that allows a user to select which electrodes of the plurality of electrodes are to be activated.

46. The ablation catheter system of claim 24, wherein the longitudinally displaceable sleeve comprises a tubular structure with longitudinal slits formed therein, with the plurality of branches being defined between the longitudinal slits.

47. The ablation catheter system of claim 24, wherein the proximal end portion comprises a partial circumferential section, while the distal end portion comprises a complete circumferential portion.

48. The ablation catheter system of claim 47, wherein the proximal end portion includes a complete circumferential section to which the plurality of branches are attached, the partial circumferential section being located proximal to the complete circumferential section.

49. An ablation catheter system comprising:
a balloon ablation catheter including an inflatable porous balloon coupled to a catheter shaft, the inflatable porous balloon includes a plurality of micropores formed therein in at least a first region of the inflatable porous balloon; and
at least one electrode movably disposed within an inside of the inflatable porous balloon.

50. The ablation catheter system of claim 49, wherein the at least one electrode is disposed within and carried by a movable hood that is rotatably coupled to a catheter shaft and configured for placement against an inner surface of the inflatable porous balloon.

51. The ablation catheter system of claim 50, wherein the hood includes at least one fluid conduit in fluid communication with an interior of the hood for delivering a conductive fluid to the interior of the hood and facilitates passage of the conductive fluid through the micropores.

52. The ablation catheter system of claim 51, wherein the at least one fluid conduit comprises an inner lumen formed in a tubular support that is attached at a distal end to the hood.

53. The ablation catheter system of claim 49, wherein the first region is a central region of the inflatable porous balloon that is between a proximal end region and a distal end region, each of the proximal end region and the distal end region being non-perforated.

54. The ablation catheter system of claim 49, wherein the at least one electrode is rotatably coupled to a catheter shaft of the balloon ablation catheter and is also movable in a radial direction both towards and away from the catheter shaft.

55. The ablation catheter system of claim 49, wherein the at least one electrode is carried by an electrode sleeve that is formed of an elastic material and is disposed within an interior of the inflatable porous balloon, wherein the electrode sleeve has a proximal end portion, an opposing distal end portion and a plurality of branches that are connected to both the proximal end portion and the distal end portion, wherein the at least one electrode comprises an electrode array that is disposed along an exterior of one or more branches of the plurality of branches and is configured for placement against an inner surface of the porous balloon.

56. The ablation catheter system of claim 55, wherein the elastic electrode sleeve is pre-formed such that in an at rest position, the plurality branches are radially deployed and expanded radially outward relative to the catheter shaft and the plurality of branches are configured such that when the porous balloon is in a delated state, the plurality of branches are retracted and moved radially inward toward the catheter shaft.
